(19) United States
(12) Patent Application Publication
(10) Pub. No.: US 2012/0124704 A1
(43) Pub. Date: May 17, 2012

(54) INFUSION OF CANNABIS

(76) Inventor: Lucas Szymanowski, San Jose, CA (US)

(21) Appl. No.: 13/215,133

(22) Filed: Aug. 22, 2011

Related U.S. Application Data
(60) Provisional application No. 61/412,779, filed on Nov. 11, 2010.

(51) Int. Cl. A01H 5/00; A61P 25/00; C12C 12/00; C12G 3/00; C12G 1/00; A61K 36/185; A23L 2/00

(52) U.S. Cl. 800/298; 424/725; 426/590; 426/592

(57) ABSTRACT

One example embodiment includes a method for infusing Cannabis. The method includes preparing the Cannabis. The method also includes placing the Cannabis in an infusible liquid. The infusible liquid is suitable for human consumption and configured to remove one or more chemical compounds from the infusible liquid.

### CROSS-REFERENCE TO RELATED APPLICATIONS

This application claims the benefit of and priority to U.S. Provisional Patent Application Ser. No. 61/412,779 filed on Nov. 11, 2010, which application is incorporated herein by reference in its entirety.

### BACKGROUND OF THE INVENTION

Cannabis, also known as hemp or marijuana, is gaining popularity around the world. It's an extremely versatile plant that's been used in various capacities for centuries. Cannabis can be used for medicinal purposes, as a recreational drug and for its fibers, which can be used as structural material, such as cordage, and cloth.

There is longstanding belief and growing scientific evidence that Cannabis has medicinal properties to fight cramps, pain, migraines, convulsions, muscle spasms, and loss of appetite and can attenuate nausea and vomiting, among other things. Cannabis has been, and is currently being, used for industrial products in construction, textiles, and paper products because of its strong fibrous properties. Cannabis is also highly nutritious and used in cuisine around the world, including Japan, Peru, Burma, Nepal and Morocco.

Cannabis is a hugely diverse species belonging to the Cannabaceae family. Humulus, commonly known as Hop, is part of the same family; like hops, Cannabis offers a variety of individual breeds each with diverse flavor and aroma profiles. The method of infusion described hereunder was developed to accentuate, heighten and emphasize the distinct properties, flavor and aroma profiles of Cannabis unique to different species, individual plants, and regional characteristics. Cannabis, like Hops, has a variety of favorable profiles which, when brought out, will appeal to different palates and allow for comparisons between said species, individual plants and the impact of yearly weather cycles on a specific plant.

Extraction of Tetrahydrocannabinol (THC), Cannabis' psychoactive ingredient, is often focused on in the art, but this does not take into account the characteristics explained above. While the method outlined in this document can and will be used to extract THC, it is not the sole purpose of the invention. Moreover, the method described hereto consists of a way to infuse a liquid medium containing ethanol with a combination of compounds which together provide a profile of the plant in totality, an effect not possible to reproduce by extraction, purification, and/or recombination of the individual components.

### BRIEF SUMMARY OF SOME EXAMPLE EMBODIMENTS

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

One example embodiment includes a method for infusing Cannabis. The method includes preparing the Cannabis. The method also includes placing the Cannabis in an infusible liquid. The infusible liquid is suitable for human consumption and configured to remove one or more chemical compounds from the infusible liquid.

Another example embodiment includes a method for infusing Cannabis. The method includes providing plant matter from Cannabis and water treating the plant matter. Water treating the plant matter includes one of soaking the plant matter in water or running water over the plant matter. The method also includes removing the plant matter from the water and placing the plant matter in an infusible liquid. The infusible liquid contains ethanol, is suitable for human consumption and is configured to remove one or more chemical compounds from the plant matter. The method further includes removing the plant matter from the infusible liquid.

Another example embodiment includes a system for allowing a user to infuse a liquid with Cannabis. The system includes pre-processed Cannabis. The pre-processed Cannabis includes plant matter from Cannabis. The plant matter has been water treated. Water treating the plant matter includes one of soaking the plant matter in water or running water over the plant matter. The plant matter is configured to be placed in an infusible liquid. The infusible liquid contains ethanol, is suitable for human consumption and is configured to remove one or more chemical compounds from the infusible liquid.

These and other objects and features of the present invention will become more fully apparent from the following description and appended claims, or may be learned by the practice of the invention as set forth hereinafter.

### BRIEF DESCRIPTION OF THE DRAWINGS

To further clarify various aspects of some example embodiments of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1 is a flowchart illustrating a method for infusing Cannabis;

FIG. 2 illustrates an example of a water treatment;

FIG. 3 illustrates an example of Cannabis infusion;

FIG. 4 illustrates an example of a liquid which has been infused with Cannabis; and

FIG. 5 illustrates an example of a system for infusing a liquid with Cannabis.

### DETAILED DESCRIPTION OF SOME EXAMPLE EMBODIMENTS

Reference will now be made to the figures wherein like structures will be provided with like reference designations. It is understood that the figures are diagrammatic and schematic representations of some embodiments of the invention, and are not limiting of the present invention, nor are they necessarily drawn to scale.

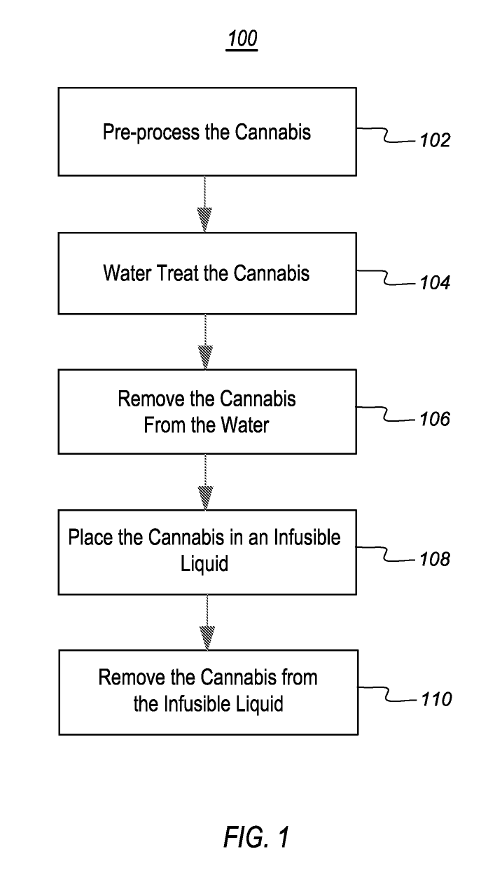

FIG. 1 is a flowchart illustrating a method 100 for infusing Cannabis. In at least one implementation, infusing Cannabis can include preparing the Cannabis and allowing one or more chemicals present in the Cannabis to permeate an infusible liquid. The resultant liquid can then be distilled, drank or used for any other desired purpose.

Cannabis is a genus of flowering plants that includes three species, Cannabis sativa, Cannabis indica, and Cannabis ruderalis. Cannabis has long been used for fiber (hemp), for seed and seed oils, for medicinal purposes, and as a recreational drug. Industrial hemp products are made from Cannabis plants selected to produce an abundance of fiber. Some hemp strains have been developed which contain minimal levels of THC (Δ9-tetrahydrocannabinol), one of the psychoactive molecules that produces the “high” associated with marijuana. The psychoactive product consists of dried flowers of plants selectively bred to produce high levels of THC and other psychoactive chemicals. Various extracts including hashish and hash oil are also produced from the plant.

Cannabis contains more than 400 different chemical compounds, including at least 66 other cannabinoids (cannabidiol (CBD), cannabinol (CBN) and tetrahydrocannabivarin (THCV), etc.). Cannabinoids are a class of chemical compounds which include the phytocannabinoids (oxygen-containing C21 aromatic hydrocarbon compounds found in the cannabis plant), and chemical compounds which mimic the actions of phytocannabinoids or have a similar structure (e.g. endocannabinoids, found in the nervous and immune systems of animals and that activate cannabinoid receptors). Different Cannabis species and different varieties within a species can have differing amounts and ratios of the chemical compounds. One of skill in the art will appreciate that species and varieties of Cannabis can be used with the method herein.

FIG. 1 shows that the method 100 can include pre-processing the Cannabis 102. In at least one implementation, pre-processing the Cannabis includes harvesting and collecting the desired Cannabis products. The portion of Cannabis which is used can include the leaves, stalks, seeds, flowers or any other portion of the plant. One of skill in the art will appreciate that different portions of the plant include different chemical compounds in different ratios, allowing a user to produce the desired ratio and concentration.

In at least one implementation, pre-processing the Cannabis 102 can begin with selecting the desired species and variety. The desired portions of the plants are then harvested from the plant. While the method described herein works with all grades of the plant, including industrial grade hemp, the best results are found when the Cannabis for infusion has been taken care of and paid attention to like grapes used in fine wines and hops used in quality beer.

One of skill in the art will appreciate that the Cannabis should be harvested carefully and in the proper ways to guarantee the best possible bouquet and profile. For example, the plant matter can be de-seeded, de-stemmed, and dried. Additionally or alternatively, the Cannabis can be ground after harvest and dried. Any preparation of the Cannabis is contemplated herein and can be used with the instant method 100 to be infused.

FIG. 1 also shows that the method 100 can include water treating the Cannabis 104. In at least one implementation, the water treatment includes placing the pre-processed Cannabis in a water bath. The water bath can be left at ambient temperature, heated or cooled to provide the proper profile to the Cannabis, as described below. Additionally or alternatively, the water treatment can include general washing, superficial debris removal, tannin extraction and non-target material extraction that would otherwise interfere with the purity of the proposed infusion method.

FIG. 1 further shows that the method 100 can include removing the Cannabis from the water 106. In at least one implementation, as much of the liquid as possible is removed. For example, a strainer, centrifuge, and/or a towel to dry the Cannabis can be used. One of skill in the art will appreciate that care should be taken not to damage the plant matter; while damaging the plant matter may not ruin the infusion process it makes the Cannabis difficult to handle. Water treating the Cannabis 104 and removing the Cannabis from the water 106 can be repeated as necessary to reach the desired Cannabis profile for infusion.

FIG. 1 additionally shows that the method 100 can include placing the Cannabis in an infusible liquid 108. For example, the ethanol can include a liquid medium containing ethanol suitable for human consumption. The types of acceptable infusible liquids containing ethanol may be combined with various other ingredients, using various production methods, including but not limited to: varieties of beer, wine, and distilled spirits containing varying amounts of ethanol. Successful infusion can take place in liquids with trace amounts of ethanol (<1% alcohol by volume—ABV) on up to those approaching 90% ABV or more. One of skill in the art will appreciate that less ethanol in the infusible liquid may lengthen the infusion process; and that the converse is true—the more alcohol present in the infusible liquid the less time the infusion process can take.

FIG. 1 shows that the method 100 can include removing the Cannabis from the infusible liquid 110. In at least one implementation, the Cannabis can be removed by way of filter or strainer. The remaining plant matter can be processed again using the method 100 to remove the remaining chemical components or be processed by other means to those known in the arts of chemical or other processing methods. Additionally or alternatively, the Cannabis infused liquid can be concentrated to increase the concentration of the infused compounds.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

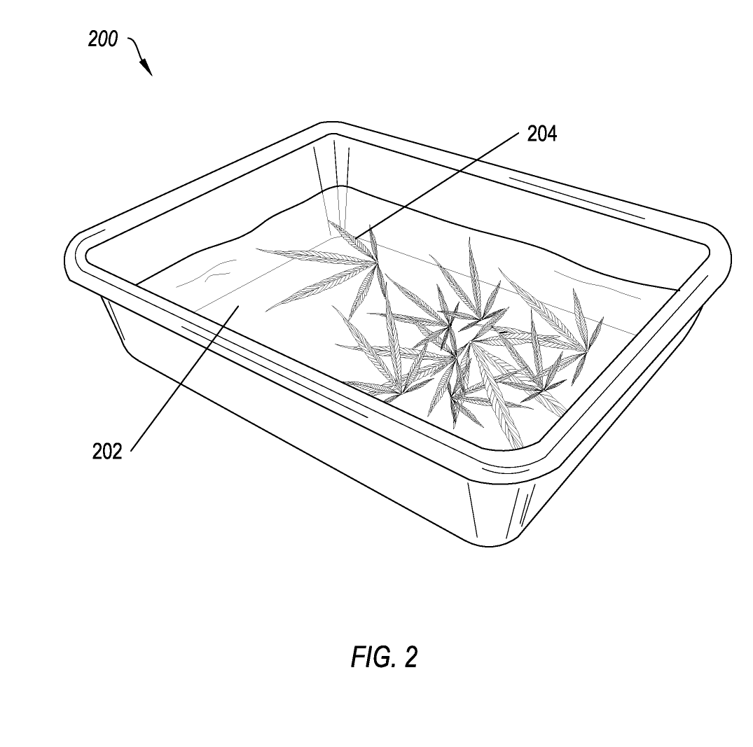

FIG. 2 illustrates an example of a water treatment 200. In at least one implementation, the water treatment 200 can be used to prepare Cannabis for infusion. For example, the water treatment 200 can be used to clean the Cannabis. Additionally or alternatively, the water treatment 200 can be used to remove one or more chemical compounds from the Cannabis, such as tannins.

A tannin (a.k.a. vegetable tannin, i.e. a type of biomolecule, as opposed to modern synthetic tannin) is an astringent, bitter plant polyphenolic compound that binds to and precipitates proteins and various other organic compounds including amino acids and alkaloids. The astringency from the tannins is what causes the dry and puckery feeling in the mouth following the consumption of unripened fruit or red wine. Likewise, the destruction or modification of tannins with time plays an important role in the ripening of fruit and the aging of wine.

FIG. 2 shows that the water treatment 200 includes water 202. In at least one implementation, if the Cannabis is being infused in a drinkable liquid or other product for human consumption then it is important to use clean sterile water 202 suitable for human consumption for this process. Clean water 202 will not to introduce new bacteria, parasites or fungi that will ruin the infusion. The water 202 can either be standing water, such as in a pot or vat, or running water that flows over the Cannabis.

FIG. 2 also shows that the water treatment 200 includes plant matter 204. In at least one implementation, plant matter 204 is placed in the water 202. The plant matter 204 can include any portion of the Cannabis plant desired. For example, the plant matter 204 can include leaves, stem, flower, seeds or any other portion of a Cannabis plant. The plant matter 204 can be from a single species and variety of Cannabis. Additionally or alternatively, the plant matter 204 can come from multiple species and/or multiple varieties of Cannabis.

In at least one implementation, the water treatment 200 can include isolating and maintaining the specific profile of the plant matter 204 being infused while removing the bulk of the tannic components that can lead to off flavors and a bitter tasting infusion. The profile is the chemical composition of the resultant infusion. Thus, the water treatment 200 of the Cannabis can remove unwanted compounds from the plant matter 204. I.e., the water treatment 200 of the plant matter 204 can leave intact the desired chemical compounds, including THC if desired, which will dissolve in the infusible liquid, as described below. Additionally or alternatively, water treatment 200 of the Cannabis can remove the woodsy taste associated with the fibrous plant matter 204.

In at least one implementation, the duration, scope, and temperature of the water treatment 200 will vary based on the desired infusion Profile. The water 202 can be from the tap, filtered, de-chlorinated, etc. and the temperature can range from just above freezing to boiling hot. Best results for tannin removal are realized when the water 202 temperature is between 130 degrees and 165 degrees Fahrenheit, although it is permissible to use water 202 below or above this temperature range. Cooler water 202 will remove less tannins, resulting in a more bitter infusion; water 202 above 165 degrees Fahrenheit runs the risk of shocking the plant matter 204 resulting in burnt flavors (which may be desirable).

One of skill in the art will appreciate that the plant matter 204 can be submerged in water 202 or the water 202 can be passed over the plant matter 204. In at least one implementation, the plant matter 204 can be kept intact during the water treatment 200. I.e., the plant matter 204 can be treated delicately as to keep the plant intact and not break it apart. Additionally or alternatively, the plant matter 204 can be agitated to break apart the plant matter 204 if so desired. One of skill in the art will appreciate that agitation may be necessary for a full wash and to ensure all the plant matter 204 is submerged if standing water 202 is used for the water treatment 200.

In at least one implementation, the duration of the water treatment 200 will vary based on the desired infusion profile. A shorter period will remove less tannins resulting in a more bitter infusion; a longer duration will remove more tannins allowing more of the profile to come through in the infusion. The minimum amount of time for water treatment 200 is based on the amount of time required to wash the plant matter 204 of dirt and other loose particles—a few minutes or less. For a less bitter infusion, the water treatment 200 can last longer. For example, the water treatment 200 can last between 30 minutes and 1 hour.

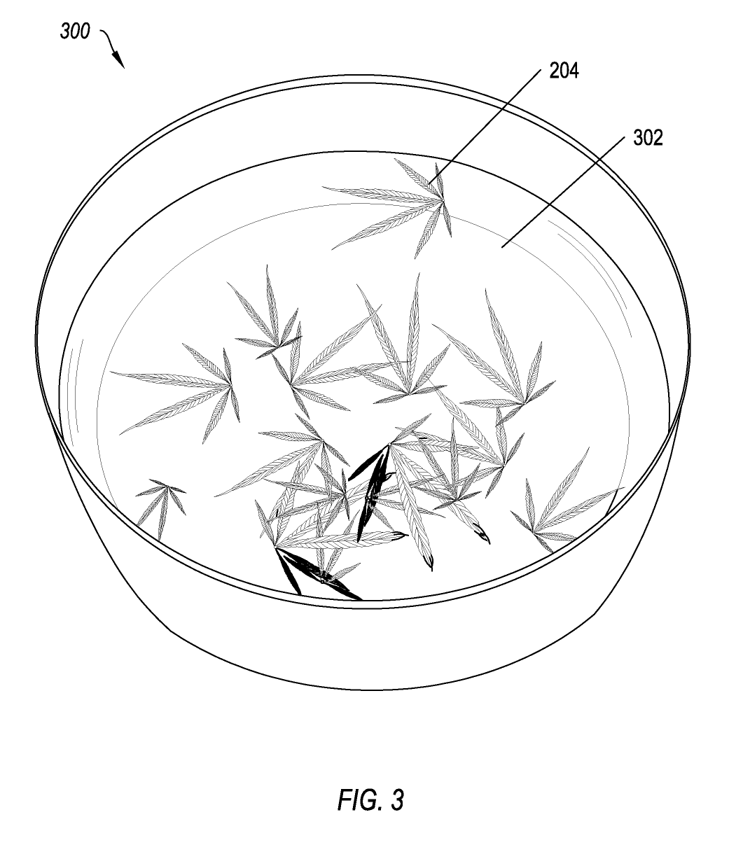

FIG. 3 illustrates an example of Cannabis infusion 300. In at least one implementation, Cannabis infusion 300 can include transferring one or more chemical compounds from the Cannabis to an infusible liquid. For example, Cannabis infusion can include transferring THC or any other compound from the Cannabis to the infusible liquid.

FIG. 3 shows that the plant matter 204 can be placed in ethanol 302. Ethanol 302, also called ethyl alcohol, pure alcohol, grain alcohol, or drinking alcohol, is a volatile, flammable, colorless liquid. Ethanol 302 is the type of alcohol found in alcoholic beverages, it is often referred to simply as alcohol or spirits. Ethanol 302 is a straight-chain alcohol, and its molecular formula is C₂H₅OH. Its empirical formula is C₂H₆O. Ethanol 302 has widespread use as a solvent of substances intended for human contact or consumption, including scents, flavorings, colorings, and medicines.

In at least one implementation, placing the Cannabis in ethanol 302 begins the infusion process. One of skill in the art will appreciate that allowing the Cannabis to sit submerged in the ethanol 302 based liquid until the desired profiles has been achieved. For example, the Cannabis can remain in the ethanol 302 until the desired THC level has been achieved or the THC level has plateaued. Additionally or alternatively, the Cannabis can remain in the ethanol 302 until the desired level of other chemical compounds has been achieved or has plateaued.

In at least one implementation, infusion of the Cannabis into the ethanol 302 should take place at standard room temperatures—between 50 degrees and 80 degrees Fahrenheit. However, one of skill in the art will appreciate that it is acceptable for the infusion to take place at lower or higher temperatures as appropriate. Gentle agitation as necessary can ensure full Cannabis immersion in the ethanol 302. One of skill in the art will appreciate that, depending on the concentration of ethanol 302, the infusion can take anywhere between a few hours to a month or more. For example, the infusion can take approximately two weeks. As used in the specification and the claims, the term approximately shall mean that the value is within 10% of the stated value, unless otherwise specified.

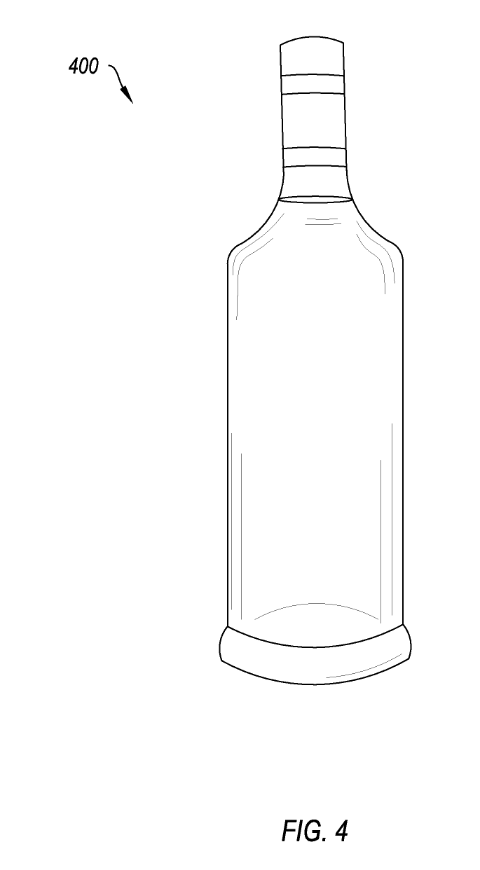

FIG. 4 illustrates an example of a liquid 400 which has been infused with Cannabis. In at least one implementation, the plant matter used to infuse the liquid 400 can be removed before the infused liquid is consumed or otherwise used. Additionally or alternatively, the plant matter, or a portion thereof, can be left in the liquid. Although the infusion would be minimal, the plant matter can be left for aesthetic purposes.

In at least one implementation, the Cannabis can be removed by way of filter or strainer. The remaining plant matter can be processed again using the method 100 to remove the remaining components or be processed by other means to those known in the arts of chemical or other processing methods. Additionally or alternatively, the Cannabis infused liquid can be concentrated to increase the concentration of the infused compounds.

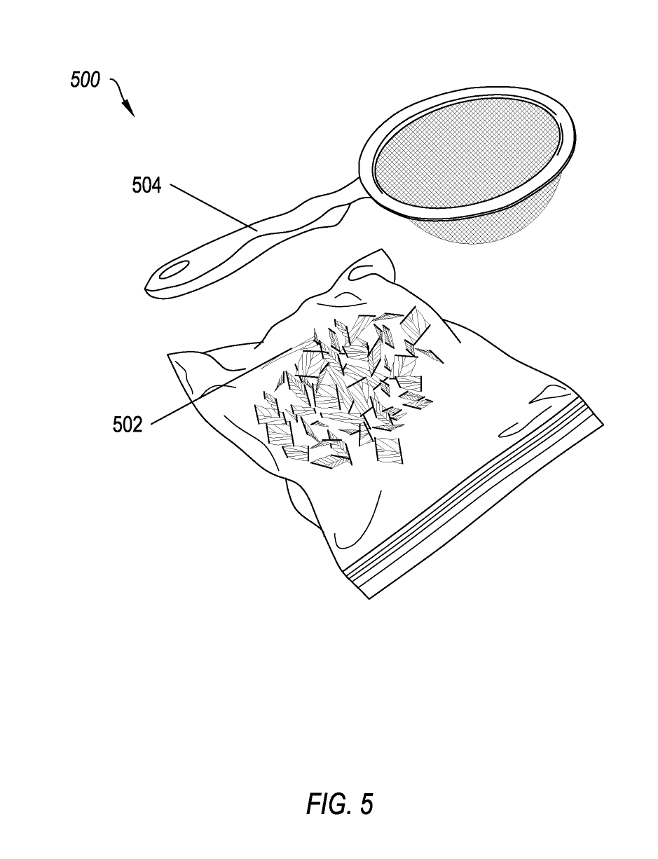

FIG. 5 illustrates an example of a system 500 for infusing a liquid with Cannabis. In at least one implementation, the system 500 can be used by a user to infuse a liquid when desired. For example, the user can add the Cannabis to an infusible liquid in a home, a restaurant or other location.

FIG. 5 shows that the system 500 can include pre-processed Cannabis 502. In at least one implementation, pre-processing the Cannabis includes harvesting and collecting the desired Cannabis products. The portion of Cannabis which is used can include the leaves, stalks, seeds, flowers or any other portion of the plant. One of skill in the art will appreciate that different portions of the plant include different chemical compounds in different ratios, allowing a user to produce the desired ratio and concentration.

In at least one implementation, pre-processing the Cannabis can begin with selecting the desired species and variety. The desired portions of the plants are then harvested from the plant. While the method described herein works with all grades of the plant, including industrial grade hemp, the best results are found when the Cannabis for infusion has been taken care of and paid attention to like grapes used in fine wines and hops used in quality beer.

One of skill in the art will appreciate that the Cannabis should be harvested carefully and in the proper ways to guarantee the best possible bouquet and profile. For example, the plant matter can be de-seeded, de-stemmed, and dried. Additionally or alternatively, the Cannabis can be ground after harvest and dried. Any preparation of the Cannabis is contemplated herein and can be used with the instant method 100 to be infused.

In at least one implementation, pre-processing the Cannabis can also include water treatment of the Cannabis. In at least one implementation, the water treatment includes placing the pre-processed Cannabis in a water bath. The water bath can be left at ambient temperature, heated or cooled to provide the proper profile to the Cannabis, as described below. Additionally or alternatively, the water treatment can include general washing, superficial debris removal, tannin extraction and non-target material extraction that would otherwise interfere with the purity of the proposed infusion method.

In at least one implementation, pre-processing the Cannabis can further include removing the Cannabis from the water. In at least one implementation, as much of the liquid as possible is removed. For example, a strainer, centrifuge, and/or a towel to dry the Cannabis can be used. One of skill in the art will appreciate that care should be taken not to damage the plant matter; while damaging the plant matter may not ruin the infusion process it can make the Cannabis difficult to handle. The water treatment of the Cannabis and removing the Cannabis from the water can be repeated as necessary to reach the desired Cannabis profile for infusion.

FIG. 5 also shows that the system 500 can include a strainer 504. In at least one implementation, the strainer 504 can be used to remove the Cannabis from the infusible liquid. I.e., the strainer 504 can allow the user to remove some or all of the Cannabis when the infusion is complete and/or when the user desires to consume or otherwise use the infused liquid. Once removed, the Cannabis can be reused by the user if so desired.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method for infusing Cannabis, the method comprising:
preparing the Cannabis; and
placing the Cannabis in an infusible liquid, wherein the infusible liquid:
is suitable for human consumption; and
configured to remove one or more chemical compounds from the infusible liquid.

2. The method of claim 1, wherein the infusible liquid includes ethanol.

3. The method of claim 2, wherein the infusible liquid includes an alcoholic beverage.

4. The method of claim 3, wherein the alcoholic beverage includes one of:
beer;
wine; or
distilled spirits.

5. The method of claim 1, wherein the ethanol is greater than 1% of the infusible liquid by volume.

6. The method of claim 5, wherein the ethanol is lower than 90% of the infusible liquid by volume.

7. The method of claim 1 further comprising removing the Cannabis from the infusible liquid.

8. The method of claim 7, wherein the Cannabis is removed from the liquid after a predetermined period of time predetermined period of time.

9. The method of claim 8, wherein the predetermined period of time is approximately two weeks.

10. The method of claim 7, wherein the Cannabis is removed after a desired concentration of THC is achieved.

11. The method of claim 7, wherein the Cannabis is removed after the concentration of THC has plateaued.

12. A method for infusing Cannabis, the method comprising:
providing plant matter from Cannabis;
water treating the plant matter, wherein water treating the plant matter includes one of:
soaking the plant matter in water; or
running water over the plant matter;
removing the plant matter from the water;
placing the plant matter in an infusible liquid, wherein the infusible liquid:
contains ethanol;
is suitable for human consumption; and
is configured to remove one or more chemical compounds from the plant matter; and
removing the plant matter from the infusible liquid.

13. The method of claim 12, wherein the plant matter includes at least one of:
leaves;
stems;
flowers; and
seeds.

14. The method of claim 12, wherein the temperature of the water is between 130 degrees Fahrenheit and 165 degrees Fahrenheit.

15. The method of claim 12, wherein the water used for the water treatment is potable water.

16. The method of claim 12 wherein water treatment includes the removal of tannin from the plant matter.

17. The method of claim 16 wherein the infusible liquid is maintained at a temperature between 50 degrees Fahrenheit and 85 degrees Fahrenheit during the infusion.

18. The method of claim 12 further comprising placing the plant matter in a second infusible liquid.

19. A system for allowing a user to infuse a liquid with Cannabis, the system comprising:
pre-processed Cannabis, wherein the pre-processed Cannabis:
includes plant matter from Cannabis, wherein the plant matter:
has been water treated, wherein water treating the plant matter includes one of:
soaking the plant matter in water; or
running water over the plant matter; and
is configured to be placed in an infusible liquid, wherein the infusible liquid:
contains ethanol;
is suitable for human consumption; and
is configured to remove one or more chemical compounds from the plant matter.

20. The system of claim 19 further comprising a strainer, wherein the strainer is configured to allow a user to remove the Cannabis.
